(19) United States
(12) Patent Application Publication
(10) Pub. No.: US 2021/0000093 A1
(43) Pub. Date: Jan. 7, 2021

(54) BOMBYX MORI HATCHING AND YOUNG SILKWORM REARING FRAME

(71) Applicant: SOOCHOW UNIVERSITY, Suzhou (CN)

(72) Inventor: Bing LI, Suzhou (CN)

(21) Appl. No.: 17/026,197

(22) Filed: Sep. 19, 2020

Related U.S. Application Data
(63) Continuation of application No. PCT/CN2018/079917, filed on Mar. 21, 2018.

(51) Int. Cl. A01K 67/04

(52) U.S. Cl. CPC A01K 67/04

(57) ABSTRACT

Disclosed is a bombyx mori hatching and young silkworm rearing frame that includes a rearing frame main body (1) and a rearing frame drawer (2), wherein the size of the rearing frame is convenient for needs of silkworm rearing production; the rearing frame drawer (2) can be used for storing silkworm eggs during hatching and adding water to moisturize during rearing; the shape and size of grooves (4) of the rearing frame drawer (2) and those of meshes of a middle meshed interlayer (3) are both designed according to physiological needs of silkworm bodies; a stacked fixing device is further arranged at the periphery of the rearing frame; and a carbonate polymer layer is arranged on the surface of the meshed interlayer (3). According to the use of the rearing frame, the labor productivity of hatching and young silkworm rearing can be improved, and a novel rearing instrument can be provided for large-scale and labor-saving silkworm rearing.

This application is a Continuation Application of PCT/CN2018/079917, filed on Mar. 21, 2018.

### TECHNICAL FIELD

The patent relates to a bombyx mori hatching and young silkworm rearing frame, and particularly relates to the preparation of a bombyx mori hatching and young silkworm rearing frame.

### BACKGROUND TECHNIQUE

Bombyx mori is an important economic insect that has been domesticated for more than 8,500 years. Bombyx mori is a completely metamorphic lepidopteran that has completely different developmental stages in morphological and physiological functions, such as eggs, larvae, pupa and adults. The intake of nutrients is mainly the feeding of mulberry leaves during larvae. Silkworm larvae can usually be divided into 5 ages, which are age 1, age 2, age 3, age 4, and age 5. Age 1-3 are called young silkworm, and age 4-5 are called old silkworm.

The young silkworm hatched from the silkworm eggs are called the newly-hatched silkworm. The process of collecting the newly-hatched silkworms for feeding is called brushing of newly hatched larva of oak silkworm. The expanding process of the young silkworms the area is called spacing, so that the operation to make the silkworm seats evenly distributed is called uniform seats. The traditional methods of collecting newly-hatched silkworms include brushing of newly hatched larvae with paper, brushing of newly hatched larvae by silkworm net, and so on. Due to the delicate body of the newly hatched newly-hatched silkworms, the individual is small and inconvenient to grasp. Generally, the newly-hatched silkworms are fed the mulberry leaves for a period of time first, and then the mulberry leaves are taken to disperse the young silkworms to achieve the spacing and uniform seats. Due to the rapid growth and development of young silkworms, the traditional methods of collecting newly-hatched silkworms are not only time-consuming and labor-intensive, but also after brushing of newly hatched larva of oak silkworm must spacing and uniform seat every day in the rearing process of the young silkworms. It is urgent to develop a new type of multifunctional feeding frame which can be used for brushing of newly hatched larva of oak silkworm.

### TECHNICAL PROBLEM

### Technical Solution

The invention develops a multifunctional of bombyx mori hatching and young silkworm rearing frame, which can improve the labor efficiency of brushing of newly hatched larva of oak silkworm and feeding young silkworm.

The invention adopts the following technical solutions:

A bombyx mori hatching and young silkworm rearing frame, comprising a frame body and a drawer; the drawer is located in the frame body; the frame body is provided with a mesh compartment; the drawer surface is provided with a groove; the mesh The bottom surface of the compartment is in contact with the surface of the drawer; the surface of the mesh spacer is provided with a carbonate polymer layer.

In the invention, the bottom surface of the frame body is sealed, and the reverse side is a sealed bottom, which increases the strength of the rearing frame. Preferably, the upper surface of the frame body is provided with a convex periphery, and the bottom surface has no convex periphery, so that the bottom surface is smaller than the surface, and a circumference around the surface is used for extracting the force of brushing of newly hatched larva of bombyx mori and the young silkworm rearing frame.

In the invention, the grooves on the surface of the drawer are evenly distributed, the grooves are used for storing silkworm eggs when brushing of newly hatched larva of oak silkworm, and moisten with water when feeding young silkworms.

In the invention, the groove is a spherical structure, which facilitates the crawling of the newly-hatched silkworm.

In the invention, the 4% to 5% of the height of the frame body is height of the drawer. It is satisfied no need for sand removal (the operation of removing the silkworm dung, the remaining mulberry leaves, etc.) for age 1.

In the present invention, on four sides of the upper surface of the frame body is provided circular holes, corresponding protrusions on four sides of the down surface. It is convenient to fix the feeding frame when it is stacked.

In the present invention, the non-inserted side of drawer is provided with a high side; the insertion side of the drawer is provided with a rib, the high side is higher than the rib. The rib is favorable for avoiding the silkworm splashing out, the high side is good for controlling the drawer.

The size of the rearing frame of the invention is just for the breeding of one box (25,000 tablets) of silkworms, and the drawers are installed under the rearing frame. The drawers can be used for storing silkworms when brushing of newly hatched larva of oak silkworm, and moisten with water when feeding young silkworms; There is a mesh compartment, the mesh shape is a square with a side length of 0.2 cm, which facilitates the hatching of the newly-hatched silkworm from the drawer layer to the upper layer; the grooves on the surface of the drawer are evenly distributed. the rearing frame of the present invention can also be used for rearing age 2-3 silkworm.

The device of bombyx mori hatching and young silkworm rearing frame composition with bombyx mori hatching and young silkworm rearing frames. The wherein the bombyx mori hatching and young silkworm rearing frame comprising frame body and drawer; the drawer is located in the frame body; the frame body is provided with a mesh compartment; the drawer surface is provided with grooves; and on four sides of the upper surface of the frame body is provided circular holes, corresponding protrusions on four sides of the down surface, the surface of the mesh compartment is provided with a carbonate polymer layer. The frame body is bottom sealing and the upper surface of the frame body is provided with a convex periphery; the grooves on the surface of the drawer are evenly distributed and have spherical structure; the 4% to 5% of the height of the frame body is height of the drawer; wherein the non-inserted side of drawer is provided with a high side; the insertion side of the drawer is provided with a rib; the high side is higher than the rib. The rib is favorable for avoiding the silkworm splashing out, the high side is good for controlling the drawer.

The silkworm eggs after hatched for 5 hours are evenly spread in the grooves on the drawer of the rearing frame, and the drawers are placed in the rearing frame, and a box of silkworms (25,000 tablets) can be used to use a rearing frame to lay the rearing frame of the silkworms. Superimposed, placed in the silkworm indoors to hatch until the next morning, then spread the superimposed feeding frame for photosensitivity, after the anterior silkworm climbed up from the mesh compartment of the rearing frame, it was raised to the mulberry newly-hatched silkworm, not Need to expand and shovel, other feeding methods are kept as usual. After the brushing of newly hatched larva of oak silkworm, the drawers can be pulled out, the hatching rate can be counted, and the drawers can be cleaned after removing the eggshells of the silkworm eggs. If necessary, an appropriate amount of water can be added in the grooves to achieve the purpose of moistening the silkworms. The rearing frame can be superimposed to cover the mulberry leaves and reduce the evaporation effect of the water. The height of the rearing frame can meet the needs of the first-year without sand removal (the operation of removing the silkworm dung, the remaining mulberry leaves and the like). One box of the second instar silkworms eggs can be used two rearing frames, and one box of the third instar silkworms eggs can be used four rearing frames (with mulberry leaves at the first time for each age). No need removed silkworm sand when the growth period. Removed silkworm sand at age 2 and 3.

In the present invention, the method preparation of Carbonate polymer layer is by, sequentially weighing MeO-PEG-OH and trimethylene carbonate and ethoxylated trimethylolpropane triacrylate and dissolved dichloromethane in a nitrogen glove box, stirred to add catalyst diphenyl phosphate; the reactor was sealed and reacted at 40° C. for 20 hours to obtain a mixed solution; then the mixture is sprayed on the surface of the mesh compartment and dried to form a carbonate polymer layer on the surface of the mesh compartment. The carbonate polymer layer facilitates the newly-hatched silkworm to climb out of the mesh barrier and avoid damage to the silkworm.

### Beneficial Effect

The invention combines the brushing of newly hatched larva of oak silkworm and feeding young silkworm in an ingenious combination, reduces the labor, improves the efficiency of the sericulture, and provides a new device for the large-scale feeding young silkworm.

### DRAWINGS

FIG. 1 is a schematic view showing the structure of bombyx mori hatching and young silkworm rearing frame;

FIG. 2 is a schematic view showing the structure of the drawer of EXAMPLE 2;

FIG. 3 is a schematic structural view of a mesh compartment of EXAMPLE 2;

FIG. 4 is a schematic view showing the structure of the groove of EXAMPLE 2;

FIG. 5 is a schematic view showing the structure of the newly-hatched silkworm and the young silkworms rearing frame of the silkworm of EXAMPLE 3;

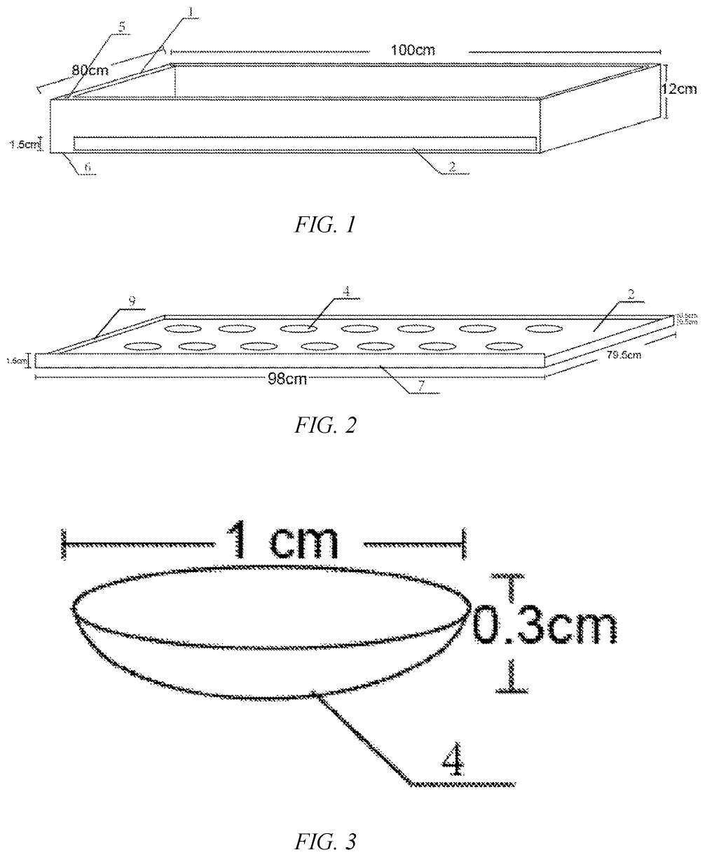

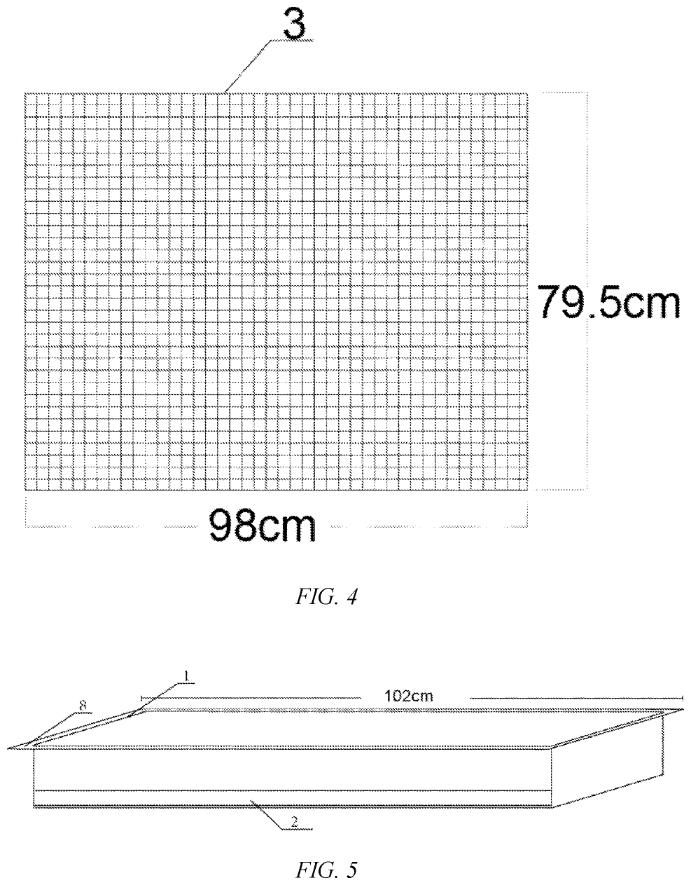

Wherein, the frame body 1; the drawer 2; the mesh compartment 3; the groove 4; the circular hole 5; the protrusion 6; the high side 7; the convex periphery 8; the rib 9.

### EXAMPLES OF THE INVENTION

### Example 1

Sequentially weighing MeO-PEG-OH and trimethylene carbonate and ethoxylated trimethylolpropane triacrylate and dissolved dichloromethane in a nitrogen glove box, stirred to add catalyst diphenyl phosphate; the reactor was sealed and reacted at 40° C. for 20 hours to obtain a mixed solution; then the mixture is sprayed on the surface of the mesh compartment and dried at 70° C. to form a carbonate polymer layer on the surface of the mesh compartment. The carbonate polymer layer was formed on the surface of the mesh compartment for the following bombyx mori hatching and young silkworm rearing frame is not shown in the drawings.

### Example 2

Bombyx mori hatching and young silkworm rearing frame, comprising frame body 1 and drawer 2; the drawer is located in the frame body; the frame body is provided with a mesh compartment 3 (a carbonate polymer layer on the surface); the drawer surface is provided with grooves4; the bottom surface of the mesh compartment is in contact with the surface of the drawer; the drawer has a drawer entrance on one side, four sides of the upper surface of the frame body is provided circular holes 5, corresponding protrusions 6 on four sides of the down surface, convenient to fix the feeding frame when it is stacked; the non-inserted side of drawer is provided with a high side 7; the insertion side of the drawer is provided with a rib 9 (on three sides). When the silkworm is planted, the silkworm species are prevented from splashing. The upper surface of the drawer is uniformly distributed with grooves, the groove is spherical, and the bottom is not transparent. It is convenient for newly-hatched silkworm to climb out when brushing of newly hatched larva of oak silkworm, and store water when cultivating silkworms; the bottom of the frame body is sealed.

The height of the frame body is 12 cm, the length is 100 cm, the width is 80 cm; the thickness of the drawer is 0.5 cm (thickness of the bottom plate), the length is 98 cm, the width is 79.5 cm, the height of the high side is 1 cm, and the rib is The height is 0.5 cm; the mesh compartment has a length of 98 cm and a width of 79.5 cm; the mesh of the mesh compartment is a square with a side length of 0.2 cm; the groove has a spherical shape with a height of 0.3 cm and a bottom diameter of 1 cm. The structure in the figure is a schematic representation, and the same components are labeled once.

### Example 3

Basic on the EXAMPLE 2, the upper surface of the frame body is provided with a convex periphery 8 having a length of 1 cm, so that the extraction is convenient, and the structural diagram is shown in FIG. 5, wherein the thickness of the frame is not marked, which does not affect the understanding of the technical solution.

### Example 4

Composition with bombyx mori hatching and young silkworm rearing frames to the device of bombyx mori hatching and young silkworm rearing frames .

The silkworm eggs after hatched for 5 hours are evenly spread in the grooves on the drawer of the rearing frame, and the drawers are placed in the rearing frame, and a box of silkworms (25,000 tablets) can be used to use a rearing frame to lay the rearing frame of the silkworms. Superimposed, placed in the silkworm indoors to hatch until the next morning, then spread the superimposed feeding frame for photosensitivity, after the anterior silkworm climbed up from the mesh compartment of the rearing frame, it was raised to the mulberry newly-hatched silkworm, not Need to expand and shovel, other feeding methods are kept as usual. After the brushing of newly hatched larva of oak silkworm, the drawers can be pulled out, the hatching rate can be counted, and the drawers can be cleaned after removing the eggshells of the silkworm eggs. If necessary, an appropriate amount of water can be added in the grooves to achieve the purpose of moistening the silkworms. The rearing frame can be superimposed to cover the mulberry leaves and reduce the evaporation effect of the water. The height of the rearing frame can meet the needs of the first-year without sand removal (the operation of removing the silkworm dung, the remaining mulberry leaves and the like). One box of the second instar silkworms eggs can be used two rearing frames, and one box of the third instar silkworms eggs can be used four rearing frames (with mulberry leaves at the first time for each age).No need removed silkworm sand when the growth period. Removed silkworm sand at age 2 and 3.

The size of the rearing frame of the invention is convenient for the production of silkworms, and the drawer of rearing frame can be used for storing silkworms during brushing of newly hatched larva of oak silkworm and adding water and moisturizing when rearing silkworms, the groove of the drawer, the size of the mesh of the mesh compartment is designed according to the physiological needs of the silkworm .There are stacked fixing devices around the rearing frame. The apply of the rearing frame can greatly improve the labor productivity of collecting ants and rearing small silkworm, and provide new silkworm tools for large-scale and labor-saving silkworm rearing.

1. A bombyx mori hatching and young silkworm rearing frame, comprising:
a frame body, and
a drawer,
wherein the drawer is located in the frame body; the frame body is provided with a mesh compartment; a surface of the drawer is provided with grooves; and a surface of the mesh compartment is provided with a carbonate polymer layer.

2. The bombyx mori hatching and young silkworm rearing frame according to claim 1, wherein the frame body is sealed at bottom; and an upper surface of the frame body is provided with a convex periphery.

3. The bombyx mori hatching and young silkworm rearing frame according to claim 1, wherein the grooves on the surface of the drawer are evenly distributed.

4. The bombyx mori hatching and young silkworm rearing frame according to claim 1, the grooves have a spherical structure.

5. The bombyx mori hatching and young silkworm rearing frame according to claim 1, wherein a height of the drawer is 4% to 5% of a height of the frame body; a non-inserted side of the drawer is provided with a high side panel; an insertion side of the drawer is provided with a rib; and the high side panel is higher than the rib.

6. The bombyx mori hatching and young silkworm rearing frame according to claim 1, wherein four corners of an upper surface of the frame body are provided with circular holes, and four corners of a lower surface are provided with corresponding protrusions.

7. The bombyx mori hatching and young silkworm rearing frame according to claim 1, wherein the carbonate polymer layer is prepared by a method comprising following steps:
sequentially weighing MeO-PEG-OH and trimethylene carbonate and ethoxylated trimethylolpropane triacrylate and dissolving in dichloromethane in a reactor in a nitrogen glove box;
adding a catalytic amount of diphenyl phosphate while stirring;
sealing the reactor and reacting at 40° C. for 20 hours to obtain a mixed solution;
spraying the mixed solution on the surface of the mesh compartment and drying to form the carbonate polymer layer on the surface of the mesh compartment.

8. A bombyx mori hatching and young silkworm rearing device comprising a plurality of bombyx mori hatching and young silkworm rearing frames, wherein each of the bombyx mori hatching and young silkworm rearing frames includes a frame body and a drawer;
the drawer is located in the frame body;
the frame body is provided with a mesh compartment;
a surface of the drawer is provided with grooves;
four corners of an upper surface of the frame body are provided with circular holes, and four corners of a lower surface are provided with corresponding protrusions; and
a surface of the mesh compartment is provided with a carbonate polymer layer.

9. The bombyx mori hatching and young silkworm rearing device according to claim 8, wherein
the frame body is sealed at bottom;
the upper surface of the frame body is provided with a convex periphery;
the grooves on the surface of the drawer are evenly distributed and have a spherical structure;
a height of the drawer is 4% to 5% of a height of the frame body; and
a non-inserted side of the drawer is provided with a high side panel; an insertion side of the drawer is provided with a rib; and the high side panel is higher than the rib.

10. The bombyx mori hatching and young silkworm rearing device according to claim 8, wherein the carbonate polymer layer is prepared by a method comprising following steps:
sequentially weighing MeO-PEG-OH and trimethylene carbonate and ethoxylated trimethylolpropane triacrylate and dissolving in dichloromethane in a reactor in a nitrogen glove box;
adding a catalytic amount of diphenyl phosphate while stirring;
sealing the reactor and reacting at 40° C. for 20 hours to obtain a mixed solution; and
spraying the mixed solution on the surface of the mesh compartment and drying to form the carbonate polymer layer on the surface of the mesh compartment.
